(19) United States
(12) Patent Application Publication
(10) Pub. No.: US 2020/0289402 A1
(43) Pub. Date: Sep. 17, 2020

(54) POUCH-TYPE ORALLY DISSOLVING FILMS WITH HIGH ACTIVE INGREDIENT CONCENTRATION

(71) Applicant: LTS LOHMANN THERAPIE-SYSTEME AG, Andernach (DE)

(72) Inventors: Christoph SCHMITZ, Rheinbrohl (DE); Marius BAUER, Andernach (DE)

(21) Appl. No.: 16/765,790

(22) PCT Filed: Nov. 21, 2018
(86) PCT No.: PCT/EP2018/082094

§ 371 Date: May 20, 2020

(30) Foreign Application Priority Data
Nov. 21, 2017 (DE) 10 2017 127 434.0

(51) Int. Cl. A61K 9/00; A61K 47/10; A61K 47/38

(52) U.S. Cl. CPC A61K 9/006; A61K 9/0056; A61K 9/009; A61K 47/38; A61K 47/10

(57) ABSTRACT

The invention relates to a dosage form for an active ingredient, to be dissolved in the oral cavity, comprising a first film layer and a second film layer that is arranged over the first film layer, wherein the composition of the first film layer can be identical to that of the second and comprises a water-soluble polymer, said first and the second film layers being interconnected by their overlapping edges so as to form at least one cavity and this cavity being filled with an active ingredient. In this configuration, the dosage form takes the form of a pouch made from two water-soluble film layers so that said film layers dissolve when the pouch is put in the mouth, and an active ingredient contained in said pouch can be released. This pouch configuration makes it possible to have a higher content of the active ingredient than comparable OTF films while avoiding thermal stress on said active ingredient during production of the dosage form. The advantageous properties of known thin-film dosage forms are substantially retained. The invention also relates to a method for producing the dosage form.

The invention relates to dosage forms for active ingredients having a cavity in which the active ingredient is present, the dosage form being water-soluble so that it is rapidly dissolved and releases the active ingredient when placed in the mouth. The fact that the active ingredient may be introduced into a cavity in the dosage form means that technical restrictions regarding the inclusion of larger active ingredient quantities in typical thin-film formulations are circumvented, and therefore larger active ingredient quantities may also be included in the dosage form without any problems. The present invention also relates to methods for producing corresponding dosage forms.

Buccal or sublingual tablets are usually used to administer active ingredients through the oral mucosa and release the active ingredient in the oral cavity. The resorption of the active ingredient through the oral mucosa offers a number of advantages over other oral dosage forms, for example the fact that the onset of action is rapid due to the bypassing of the gastrointestinal passage and the fact that the active ingredient utilisation is high.

Another problem with tablets or capsules is that they are usually swallowed, which means that the patient has to take a liquid with which he can ingest this dosage form. Sometimes, however, older patients or children have difficulty swallowing, and therefore they refuse to take tablets or capsules or are reluctant to take them. In addition, it is possible that tablets and capsules are kept in the mouth for a long time and then spat out by the patient. This often results in poor compliance, which is detrimental to the progress of healing or the success of the therapy.

An alternative dosage form to the known buccal and sublingual tablets is known in the form of flat-form-oblate-like dosage forms, also known as wafers. For example, U.S. Pat. No. 5,529,782 describes a rapidly soluble film product made of soluble polymer material or complex polysaccharides, which is mainly used for the administration of contraceptives. The film product should have a thickness of 3 to 4 mm and its solubility should be settable so that it has dissolved within 5 to 60 seconds after administration. The film product may also be in the form of a laminate which has gas-foamed cavities.

EP 0 450 141 B1 describes a carrier material for the administration of pharmaceuticals which dissolves rapidly upon contact with saliva. This carrier material is a porous, dehydrated, skeletal carrier material, especially based on proteins and polysaccharides. The cavities created by dehydration are used for the introduction of liquid active ingredients.

In WO 00/18365 an edible film is proposed which dissolves rapidly, but also can adhere well to the oral mucosa in order to release antimicrobial substances and reduces the number of unwanted microorganisms in the oral flora. The antimicrobial substances are, for example, essential oils which are preferably mixed as lipophilic phase with pullulan as matrix material in the aqueous phase.

WO 02/02085 describes rapidly disintegrating dosage forms for the release of active ingredients in the oral cavity or other bodily openings, the dosage form having a matrix which contains at least one water-soluble polymer as basic substance and which is provided with cavities.

Oral films (OTF (=oral thin film) systems) that dissolve rapidly in the oral cavity must be formulated so that the film meets certain physical requirements. For example, such films must have a certain minimum strength so that they do not break when handled by the patient. A further problem with OTF formulations is that the films cannot be produced in any thickness, since the essential property of the films is that they dissolve rapidly in the mouth. However, this is no longer guaranteed in the case of relatively thick films, since the entry of water or saliva into the inner area of the film is more difficult with a greater thickness.

In addition, OTF systems are limited not only in terms of their thickness but also in terms of their maximum size, since the user must be able to put the film in the mouth and on the tongue without any problems; this would no longer be possible with very large films. Due to these general conditions, the amount of active ingredient to be applied is limited to about 20 mg for normal film-like OTF formulations.

On the one hand this is a problem with active ingredients that have to be applied in higher quantities, but on the other hand it is also a problem with bitter or other active ingredients that are perceived as having an unpleasant taste, since these generally have to be formulated with significant quantities of taste-masking agents. Even in this case, however, the total amount of the active ingredient and any additional taste-masking agents in an OTF formulation is limited to about 20 mg.

Against this background, there is a need for a dosage form of active ingredients which offers the same advantages as the known OTF formulations, i.e. in particular rapid absorption and release of the active ingredient in the oral cavity, but which is not subject to such severe restrictions in terms of the possible active ingredient quantity to be administered.

A further problem with the known OTF systems is that, in order to produce the films, active ingredients have to be mixed with the matrix material used, for which purpose either a solvent may be used or mixing is carried out within the scope of an extrusion process. When processing with the aid of solvents, this solvent must be removed from the system during the further course of the process, for which purpose the system is usually heated. This poses a problem for active ingredients that are unstable at high temperatures, since the active ingredients integrated into the OTF decompose during the evaporation of the solvent. Alternatively, the solvent may also be removed under a slight vacuum. However, this requires suitable equipment and, from a technical point of view, may only be carried out at greater expense, which entails cost disadvantages.

In an extrusion process the active ingredients are also exposed to a higher temperature, which may lead to a partial decomposition of the active ingredient.

Against this background, there is also a need for a dosage form for an active ingredient that can be produced without having to expose the active ingredient to high temperatures. The aim is to produce dosage forms which can also be loaded with temperature-labile active ingredients.

The present invention addresses this need.

To achieve the aim described above, the present invention proposes, according to claim 1, a dosage form for an active ingredient to be dissolved in the oral cavity, comprising a first film layer and a second film layer that is arranged over the first film layer, wherein the composition of the first film layer can be identical to that of the second layer and comprises a water-soluble polymer, the first and second film layers being interconnected by their overlapping edges so as to form at least one cavity, and the cavity being filled with an active ingredient.

Accordingly, the dosage form according to the invention comprises substantially a pouch or bag, which is formed by two film layers arranged one over the other and which is formed by the connection of the film layers in the edge area. An active ingredient may then be introduced into the cavity of the pouch or bag. Since the two film layers comprise water-soluble polymers similarly to regular OTF formulations, they exhibit similar dissolution properties compared to regular OTF formulations. Compared to these, however, the dosage forms according to the invention offer the advantage that the active ingredient can be introduced only after the films have dried, so that a direct thermal stress of the active ingredient, for example as a result of the drying of the films, is avoided.

The terms “pouch” and “bag” have synonymous meanings in the context of the following description.

The term “two layers of film arranged one over the other” includes both embodiments in which two separate film layers are arranged one over the other, as well as embodiments which are created by positioning two film layers one over the other by the folding of a single film.

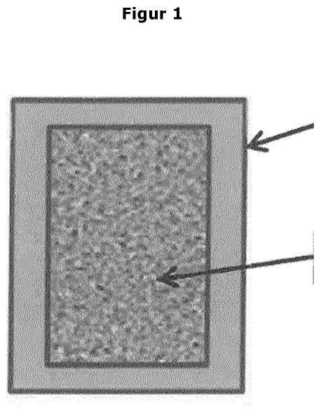

An example of a dosage form according to the invention is shown in FIG. 1, in which 1 denotes the edge region by which the two film layers are interconnected, while 2 shows the cavity filled with active ingredient.

The fact that the dosage form is formed as a “water-soluble” pouch means that much larger quantities of active ingredient and/or additional excipients may also be introduced into the cavity. A final sealing of the pouch after the insertion of the active ingredient may be carried out over only one edge of the dosage form, for which purpose a sealing only has to be carried out in the edge area of the dosage form, such that the active ingredient situated in the middle of the dosage form does not have to be exposed to any direct thermal stress.

In the context of the present invention, a “water-soluble polymer” means water-soluble and/or water-swellable polymers which rapidly dissolve and disintegrate in moist and aqueous environments, such as the oral cavity, thus releasing an active ingredient incorporated in the dosage form.

The statement that the first and second film layers are “interconnected by their overlapping edges to form at least one cavity” is to be understood to mean that the first and second film layers are in contact with each other in the region of their surface (provided that the cavity is not filled) but are not joined together in this region, so that the two film layers may be separated from each other in this region by the introduction of a material (especially the active ingredient) without effort. The statement also includes round embodiments of the film layers, although in this case only one overlapping edge is present, which, however, is not connected over its entire circumference in order to facilitate the filling with an active ingredient.

The “cavity” contains the active ingredient but is substantially free of water-soluble polymer. In addition, the cavity preferably does not contain a continuous formulation of the active ingredient, which is in contact with the full surface of the first and second film layers, but contains the active ingredient in a form in which discrete gas spaces are present between individual active ingredient particles.

The connection of the first film layer to the second film layer may be achieved by gluing or sealing. When gluing, for example, a suitable adhesive may be introduced into the gap between the first and second film layer, thus fixing the first film layer to the second film layer. For sealing, the first film layer and the second film layer may be heated and pressed against each other so that the first film layer adheres to the second film layer in the sealing area.

An example of a suitable water-soluble adhesive for connecting the adhesive layers is Plastoid E35H (softened Eudragit E100; added modifiers are lauric acid, adipic acid and glycerol).

Another suitable water-soluble adhesive is an adhesive based on at least one water-soluble polymer and at least one plasticiser, the water-soluble polymer preferably being shellac, a vinylpyrrolidone/vinyl acetate copolymer, a polyvinyl caprolactam/polyvinyl acetate/polyethylene glycol copolymer, hydroxypropyl cellulose or hydroxypropyl methylcellulose and/or polyvinylpyrrolidone. Glycerol, polyethylene glycol, especially polyethylene glycol 200, sorbitol and/or tributyl citrate are suitable plasticisers for combination with the water-soluble polymer. The preferred plasticiser is preferably selected from glycerol, polyethylene glycol 200 and/or tributyl citrate.

With regard to the ratio of water-soluble polymer to plasticiser, the adhesive is not subject to any relevant restrictions as long as the ratio is set in such a way that the mixture is sufficiently tacky and workable. A favourable mixing ratio may be a ratio of water-soluble polymer to plasticiser of about 85 to 50 to about 15 to 50, preferably 85 to 65 to about 15 to 35, more preferably about 80 to 60 to about 20 to 40, even more preferably about 80 to 50 to about 20 to 50, even more preferably about 82 to 68 to about 18 to 32 and most preferably about 80 up to 70 to about 20 to 30.

It has already been mentioned above that the composition of the first film layer can be identical to that of the second film layer. Since this leads to a simplification of the production of the dosage form according to the invention, it is preferred within the scope of the present invention if the composition of the first film layer and the second film layer is identical.

On the other hand, in certain cases it may be useful if the first film layer and the second film layer are based on different compositions. For example, it may be desirable to form one of the film layers as a mucoadhesive layer, while the second layer is relatively quickly soluble in an aqueous environment, thus releasing the active ingredient. In another embodiment, it may be expedient if the first film layer is a mucoadhesive film layer and the second film layer dissolves more slowly in the oral cavity than the first film layer.

With regard to the form of the active ingredient, the present invention is not subject to any significant limitations. Thus, the active ingredient may be in liquid or solid form, with powdery, granular, micro- or nano-particulate or micro- or nano-encapsulated forms being especially expedient as solid forms. If the active ingredient is in liquid form, it is mostly not in aqueous solution or suspension, however, as this impairs the integrity of the surrounding film layers. If the active ingredient is in liquid form, this form to the greatest possible extent should not affect the adjacent film layers. Active ingredients in solid form are preferred, and for solid formulations those based on lipophilic base materials, in which the active ingredient is dissolved or dispersed, should be excluded where possible. Accordingly, in a preferred embodiment the active ingredient is not present in the form of a lipophilic or oily or waxy formulation.

The term “micro-particulate” is understood in the context of the present invention to mean a material in which 90 wt. % and preferably 95 wt. % of the particles have a particle size in the range of less than 1 mm to 1 μm. The term “nano-particulate” is understood in the context of the present invention to mean a material in which 90 wt. % and preferably 95 wt. % of the particles have a particle size in the range of less than 1 μm.

In the terms “micro- or nano-encapsulated”, the above statement refers to the encapsulated particles.

As indicated above, the main advantage of the dosage form according to the invention is that it also allows the inclusion of relatively large active ingredient-containing fillings. For example, it is preferred if the dosage form has a quantity of active ingredient-containing filling which is greater than about 20 mg and especially greater than about 30 mg.

An appropriate upper limit for the content of the active ingredient-containing filling can be specified as a quantity of 1000 mg. An upper limit of 500 mg is preferred, 200 mg is more preferred, and 100 mg is preferred further still.

In an embodiment with thermoformed films or large pockets, the quantity of active ingredient filling may also be even greater. The quantities are then dependent not only on the size of the pocket, but also on the depth of the cavity produced by thermoforming.

A quantity of about 50 to about 200 mg can be specified as an especially favourable range for the active ingredient-containing filling.

The size of the dosage form according to the invention is expediently dimensioned to receive an appropriate amount of active ingredient-containing filling. As a rough guideline, a surface area in the range of about 1 to about 10 cm², and preferably about 1.5 to about 6 cm²′ can be specified. If the dosage form is, for example, in the form of a rectangular pouch, these dimensions may be about 2×2.5 cm or about 1×1.5 cm.

The dosage form according to the invention is generally of thin and flat or slightly curved design, for example in the form of small pouches, bags, sachets, packets or pads. These small pouches, bags, sachets, packets or pads may have various geometric shapes, for example circular, elliptical, oblong or polygonal, such as especially, rectangular or square.

The thickness of the film layers is preferably about 0.01 to about 2 mm, especially preferably in the range of about 0.02 to about 0.5 mm.

With respect to the water-soluble polymer, the present invention is not subject to any relevant limitations, on the proviso that the water-soluble polymer should be a pharmaceutically acceptable material. Suitable water-soluble polymers are, for example, starch and starch derivatives, dextrans; cellulose derivatives, such as carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose; polyacrylic acid, polyacrylates, polyvinylpyrrolidones, polyvinyl alcohol, polyethylene oxide polymers, polyacrylamides, polyethylene glycol, gelatin, collagen, alginates, pectins, pullulan, tragacanth, chitosan, alginic acid, arabinogalactan, galactomannan, agar, agarose, carrageenan and natural gums. Especially preferred in the context of the present invention are water-soluble polymers selected from the group comprising polyvinyl alcohol, polyethylene glycol, polyethylene oxide, cellulose derivatives, pullulan, gelatin and agar. Mostly preferred in the context of the present invention is polyvinyl alcohol as a water-soluble polymer.

The proportion of water-soluble polymer in the first and second film layer is usually about 85 to about 100 wt. %, especially about 90 to about 99.9 wt. % and most preferably about 95 to about 99.5 wt. %. Since the polymer film does not have to contain any active ingredient, unlike conventional OTF systems, the proportion of water-soluble polymer may be very high. On the other hand, depending on the intended application result, additives such as taste-masking agents or part of the active ingredient may be incorporated in the first film layer and/or in the second film layer. In this case, the proportion of water-soluble polymer in the first film layer and in the second film layer may be less than that indicated above, but it should still be in the range of about 15 to about 75 wt. % and preferably about 50 to about 70 wt. %.

The active ingredient may be, in principle, any active ingredient suitable for oral administration, with pharmaceutical active ingredients being preferred. Pharmaceutical active ingredients which are suitable for oral applications in the context of the present invention are, for example, anti-allergic agents, anti-arrhythmic agents, antibiotics, anti-diabetic agents, anti-epileptic agents, antihistamines, antitussives, cardiotonic agents, diuretics, anti-hypertensive agents, anaesthetics, nerve muscle blockers and sex hormones, such as vasopressors. Specific examples are acetaminophen, adrenalin, alprazolam, amlodipine, anastrozole, apomorphine, aripiprazole, atorvastatin, baclofen, benzocaine, benzocaine/menthol, benzydamine, buprenorphine, buprenorphine/naloxone, buprenorphine/naloxone/cetirizine, cetirizine, chlorpheniramine, clomipramine, dexamethasone, dextromethorphan, dextromethorphan/phenylephrine, diclofenac, diphenhydramine, diphenhydramine/phenylephrine, donepezil dronabinol, epinephrine, escitalopram, famotidine, fentanyl, glimepiride, GLP-1 peptides, granisetron, insulin, insulin nanoparticles, insulin/GLP-1 nanoparticles, ketoprofen, ketotifen, caffeine, levocetirizine, loperamide, loratadine, meclizine, methylphenidate, midazolam, mirodenafil, montelukast, multimeric-001, naloxone, nicotine, nitroglycerine, olanzapine, olopatadine, ondansetron, oxybutynin, pectin, pectin/menthol, pectin/ascorbic acid, PediaSUNAT (artesunate and amodiaquine), piroxicam, phenylephrine, prednisolone, pseudoephedrine, risperidone, rivastigmine, rizatriptan, selegiline, senna glycosides, sildenafil citrate, simethicone, sumatriptan, tadalafil, testosterone, triamcinolone acetonide, triptan, tropicamide, voglibose, zolmitriptan, zolpidem, or pharmaceutically acceptable salts of these compounds. As non-pharmaceutical active ingredients, the dosage form according to the invention may contain, for example, active ingredients for oral hygiene, such as menthol. The pharmaceutically active ingredient may also be a mixture of different active ingredients.

The dosage form according to the invention may contain, in addition to the already mentioned water-soluble polymer as a component of the first and second film layer and an active ingredient which is located in the cavity between the first and second film layers, further ingredients, especially auxiliaries, which are selected from the group comprising dyes, aromatic substances, especially flavourings and/or odorous substances, sweeteners, taste-masking agents, surfactants, enhancers, pH regulators, preservatives and/or antioxidants. The above-mentioned auxiliaries may be a component of one or both film layers and/or may be introduced together with the active ingredient into the cavity between the two film layers.

The addition of flavourings, odorous substances and aromatic substances, individually or in combination, is especially advantageous.

One taste-masking agent is an ion exchange resin.

Ion exchange resins which are preferred for use in the dosage form according to the invention are water-insoluble and consist of a pharmacologically inert organic or inorganic matrix containing covalently bonded functional groups which are ionic or can be ionised under suitable pH value conditions. The organic matrix may be synthetic (for example polymers or copolymers of acrylic acid, methacrylic acid, sulphonated styrene, sulphonated divinylbenzene) or partially synthetic (for example modified cellulose and dextrans). The matrix may also be inorganic, for example silica gel, modified by the addition of ionic groups.

The covalently bonded ion groups may be strongly acidic (for example sulphonic acid), weakly acidic (for example carboxylic acid), strongly basic (for example quaternary ammonium), weakly basic (for example primary amine) or a combination of acidic and basic groups. In general, those types of ion exchangers which are suitable for use in ion exchange chromatography and for applications such as the deionisation of water are suitable for use in the dosage forms according to the invention.

The ion exchange resin is preferably a resin based on crosslinked polystyrene. The polystyrene is crosslinked with a crosslinking agent selected from difunctional compounds capable of crosslinking polystyrenes. The crosslinking agent is preferably a divinyl or polyvinyl compound. Most preferred is the crosslinking agent divinylbenzene.

In general, the polystyrene is expediently crosslinked to an extent of about 3 to about 20%, preferably about 4 to about 16%, more preferably about 6 to about 10%, and most preferably about 8 wt. %, based on the total polystyrene. The polystyrene is crosslinked with the crosslinking agent by known means.

Ion exchange resins especially suitable as taste-masking agents within the scope of the present invention have exchange capacities below about 6 milliequivalents per gram (meq/g) and preferably below about 5.5 meq/g.

The size of the ion exchange resin particles should preferably fall within the range of about 20 to about 200 micrometres. Particle sizes well below the lower limit are difficult to handle in alien processing steps. Particle sizes above the upper limit, for example commercially available ion exchange resins of spherical shape and diameters up to about 1000 micrometres, are gritty in liquid dosage forms and have a stronger tendency to break when exposed to dry hydration cycles.

Representative resins useful in this invention comprise AMBERLITE IRP-69 (available from Dow Chemical) and Dow XYS-40010.00 (available from Dow Chemical). Both are sulphonated polystyrene polymers, crosslinked with 80 divinylbenzene, with an ion exchange capacity of about 4.5 to 5.5 meq/g dry resin (H±form). Their main difference is their physical form. AMBERLITE IRP-69 comprises irregularly shaped particles with a size range of 47 to 149 micrometres, produced by milling the upper, large-area spheres of AMBERLITE IRP-120. The Dow XYS 40010.00 product comprises spherical particles with a size range of 45 to 150 micrometres. Another useful exchange resin, Dow XYS-40013.00, is a polymer consisting of polystyrene crosslinked with 8% divinylbenzene and functionalised with a quaternary ammonium group. Its exchange capacity is normally in the range of about 3 to 4 meq/g dry resin. Another suitable resin is AMBERLITE IRP-64.

A taste-masking agent may be present as a component of the first and/or second film layer, but may also be introduced into the cavity of the dosage form according to the invention, as shown above. If the taste-masking agent is an ion exchange resin, it should be noted that this is only effective if the active ingredient is dissolved in the presence of the ion exchange resin. Therefore, it is not possible to achieve a taste-masking effect for example with formulations in which an ion exchange resin is formulated as a component of the first or second film layer whereas the active ingredient is introduced into the cavity of the dosage form according to the invention. Ion exchange resins as taste-masking agents should therefore be formulated in the same component of the dosage form according to the invention as the active ingredient, the active ingredient being bonded to the ion exchange resin expediently by an ionic bond.

If the dosage form according to the invention contains a taste-masking agent, this may be incorporated into one or more of the film layers, or, in the case of a multi-layer film structure, into one or more outer layers of the film. This allows, for example, an early release of the taste-masking agent from an outer polymer layer as compared to the release of the active ingredient, so that the taste receptors, for example for bitter-tasting active ingredients inside the dosage form according to the invention, may be blocked already before the active ingredient is released.

In addition, the first and/or the second film layer may contain at least one pigment or UV-absorbing agent which protects a light-sensitive active ingredient introduced into the cavity of the dosage form against UV light. In addition, it is expedient if the first and/or the second film layer contains one or more dyes, flavourings or sweeteners.

In addition to the auxiliaries already mentioned above, the first film layer and/or the second film layer may also contain other components to optimise their flexibility or other physical properties, such as at least one plasticiser and/or one humectant. Preferred plasticisers and/or humectants in the context of the present invention are selected for example from the group comprising glycerol, propylene glycol, polyethylene glycol and citric acid esters.

In addition, the first film layer and/or the second film layer may be embodied as a foam, i.e. may contain an introduced gas, such as air, nitrogen or CO₂, or another gas.

As indicated above, the first film layer and the second film layer may be formed with one or more layers in the dosage form according to the invention, it also being possible for the first film layer and/or the second film layer to be constructed of several layers of the same composition, for example by producing the first film layer or the second film layer by applying the composition in layers one above the other. On the other hand the layers may differ in their composition, for example by introducing a pigment or a UV-absorbing agent into a layer of the first film layer or second film layer and by overlaying or underlaying a composition without pigments or UV-absorbing agent.

In a multi-layer construction, one or more of the layers may be embodied as a foam, i.e. may contain an introduced gas, such as air, nitrogen or CO₂, or another gas.

Furthermore, the dosage form according to the invention may also be designed in such a way that it has two spatially separated cavities. Thus, for example, the first film layer may be connected to the second film layer by an additional sealing in the region of its surface. In this way, two or more cavities having the same or different fillings may be formed between the film layers.

This is especially advantageous when a base which is not storage-stable as such, but is mucosa-compatible is used as active ingredient, while the salt of the active ingredient has improved storage stability, but is not mucosa-compatible. In this case, it is possible to introduce the salt of the active ingredient into a first cavity of the dosage form according to the invention and to introduce an auxiliary base into a second cavity of the dosage form according to the invention, wherein, upon introduction of the dosage form into the oral cavity, the auxiliary base and the salt of the active ingredient are released so that the base of the active ingredient, which is not storage-stable but mucosa-compatible, may be formed. A connection of the first and the second film layer in the region of their surface may also be made by a peeling seam which is separated by massaging the dosage form before use and thus allows a mixing of the filling of the first cavity with the filling of the second cavity.

In addition, in order to increase the volume between the two film layers of the dosage form according to the invention, it is possible for the first film layer and/or the second film layer to have a non-planar form. For this purpose, the first film layer or the second film layer preferably may be thermoformed to obtain more fill volume with the same base area.

Furthermore, it is possible that the dosage form according to the invention not only contains active ingredient introduced into the cavity, but also active ingredient introduced into the film layers. Finally, it is possible that the active ingredient introduced into the cavity is introduced in various modifications, for example one part in a directly-releasing form, while another part is introduced in granulated form or in a delayed-releasing form, in order to produce a mixed kinetics of the release of the active ingredient.

The dosage form according to the invention is especially suitable for oral administration of active ingredients, including buccal, gingival or sublingual administration, or administration to the palate.

Finally, another aspect of the present invention relates to a method for producing a dosage form of the type described above, the method comprising the following steps:
a) positioning a first and a second film layer one over the other,
b) fastening the first film layer to the second film layer in such a way that at least one pouch is formed between the first film layer and the second film layer,
c) if necessary, cutting the film double layers obtained in b) to obtain individual pouches,
d) filling the at least one pouch with an active ingredient, and
e) closing the pouch(es).

The fastening in step b) and/or the closing of the pouch (in step e)) are/is preferably achieved by gluing or sealing within the scope of this method.

The positioning of a first film layer and a second film layer one over the other in step a) may be done either by positioning two individual films one over the other or by folding a film in its middle so that two film layers arranged one over the other which are connected at one edge are formed.

In the following, the present invention will be more closely illustrated by means of a few examples, which, however, are not to be interpreted as definitive for the scope of protection of the application.

### EXAMPLE 1

Polymer films with the compositions given in Table 1 were formulated and provided with a filling, as shown in Table 1. For this purpose, the various polymer films were first coated from solutions of the listed ingredients with the help of a coating box, which were dried to a film. Afterwards, corresponding film pieces were punched out, and a double layer with the dimensions given in Table 1 was created by folding. The double layer thus produced was then heat-sealed together on two of the edges of the film to create a pouch. The filling was then filled in, and the resulting pouch was also heat-sealed at the open edge.

TABLE 1

| | A | | B | | C | |
|---|---|---|---|---|---|---|
| Polymer film 1 | | | | | | |
| Polyvinyl alcohol | 96.9% | | 96.9% | | 96.9% | |
| Dye | 0.1% | | 0.1% | | 0.1% | |
| Flavouring | 1% | | 1% | | 1% | |
| Sweetener | 2% | | 2% | | 2% | |
| Weight per unit area | 48 | g/m² | 48 | g/m² | 48 | g/m² |
| Polymer film 2 | as polymer | | as polymer | | as polymer | |
| | film 1 | | film 1 | | film 1 | |
| Filling | | | | | | |
| Amberlite IPR-64 | 96% | | | | | |
| Flavouring | 1% | | | | | |
| Sweetener | 3% | | | | | |
| Miglyol | | | 100% | | 33% | |
| Lactose | | | | | 66% | |
| Filling quantity | 100 | mg | 70 | μl | 150 | mg |

| | | | |
|---|---|---|---|
| Pouch size | 20 × 25 mm | 20 × 25 mm | 20 × 25 mm |

In example C, miglyol was formulated as a liquid model active ingredient with lactose as a binder. This facilitates sealing to form a completely closed pouch, since the binder prevents liquid material from entering the region of the seal. The binder may thus prevent the liquid material from weakening the seal.

### EXAMPLE 2

The following dosage forms containing dextromethorphan were produced in the same way as described in Example 1. The compositions of these dosage forms are shown in Table 2 below.

TABLE 2

| | A | | B | | C | |
|---|---|---|---|---|---|---|
| Polymer film 1 | | | | | | |
| Polyvinyl alcohol | 96.9% | | | | | |
| Polyol N10 | | | 95.9% | | | |
| Pullulan | | | | | 95.6% | |
| Xanthan | | | | | 0.3% | |
| Dye | 0.1% | | 0.1% | | 0.1% | |
| Flavouring | 1% | | 1% | | 1% | |
| Sweetener | 2% | | 3% | | 3% | |
| Weight per unit area | 45 | g/m² | 45 | g/m² | 45 | g/m² |
| Polymer film 2 | as polymer | | as polymer | | as polymer | |
| | film 1 | | film 1 | | film 1 | |
| Filling | | | | | | |
| Dextromethorphan/ | 96% | | 96% | | 96% | |
| Amberlite 64 1:1 | | | | | | |
| Flavouring | 1% | | 1% | | 1% | |
| Sweetener | 3% | | 3% | | 3% | |
| Filling quantity | 52 | mg | 52 | mg | 52 | mg |

| | | | |
|---|---|---|---|
| Pouch size | 20 × 25 mm | 20 × 25 mm | 20 × 25 mm |

1. A dosage form for an active ingredient to be dissolved in the oral cavity, comprising a first film layer and a second film layer that is arranged over the first film layer, wherein the composition of the first film layer can be identical to that of the second film layer and comprises a water-soluble polymer, the first and second film layers being interconnected by their overlapping edges to form at least one cavity, and the cavity being filled with an active ingredient.

2. The dosage form according to claim 1, characterised in that the first and second film layers are glued or sealed together by their overlapping edges.

3. The dosage form according to claim 1, characterised in that the compositions of the first and second film layers are identical.

4. The dosage form according to claim 1, characterised in that the first film layer is formed as a mucoadhesive film layer and the second film layer dissolves more slowly in the oral cavity than the first film layer.

5. The dosage form according to claim 1, characterised in that the active ingredient is present in liquid, powdery, granular, micro- or nano-particulate, or micro- or nano-encapsulated form in the cavity of the dosage form.

6. The dosage form according to claim 1, characterised in that the dosage form contains an active ingredient-containing filling in a quantity of 50 to 1000 mg.

7. The dosage form according to claim 1, characterised in that the water-soluble polymer in the first and second film layer is selected from the group comprising polyvinyl alcohol, polyethylene glycol, polyethylene oxide, celluloses, pullulan, gelatin and agar.

8. The dosage form according to claim 1, characterised in that it comprises at least one auxiliary selected from the group comprising dyes, flavourings, sweeteners, taste-masking agents, surfactants, enhancers, pH regulators, preservatives and/or antioxidants, as a component of one or both film layers or of the cavity.

9. The dosage form according to claim 8, characterised in that it contains a taste-masking agent as part of the first and/or second film layer.

10. The dosage form according to claim 1, characterised in that the first and/or the second film layer contains at least one pigment and/or a UV-absorbing agent.

11. The dosage form according to claim 1, characterised in that the first and/or the second film layer is/are formed with one or more layers.

12. The dosage form according to claim 1, characterised in that the first and/or the second film layer contain at least one plasticiser and/or a humectant.

13. The dosage form according to claim 1, characterised in that the first and/or the second film layer are interconnected in the region of their surface by means of an additional seal, so that at least two cavities are formed between the film layers, and wherein the cavities have identical or different fillings.

14. The dosage form according to claim 1, characterised in that the first and/or the second film layer has a non-planar form.

15. A method for producing a dosage form wherein the method comprises the following steps:
a) positioning a first and a second film layer one over the other,
b) fastening the first film layer to the second film layer in such a way that at least one pouch is formed between the first film layer and the second film layer,
c) if necessary, cutting the film double layers obtained in b) to obtain individual pouches,
d) filling the at least one pouch with an active ingredient, and
e) closing the pouch(es).

16. A method according to claim 15, wherein the fastening in step b) and/or the closing of the pouch(es) in step e) are/is achieved by gluing or sealing.

17. The dosage form according to claim 12, characterised in that the at least one plasticiser and/or humectant is selected from the group consisting of glycerol, propylene glycol, polyethylene glycol and citric acid ester.

18. The dosage form according to claim 1, characterised in that the first and/or the second film layer is in the form at a deep-drawn film.
